(19) United States
(12) Patent Application Publication
(10) Pub. No.: US 2024/0164425 A1
(43) Pub. Date: May 23, 2024

(54) LACTOBACILLUS FERMENTATION COMPOSITION CONTAINING MUSHROOM EXTRACT AND GELATIN HYDROLYZATE AND PREPARATION METHOD THEREOF

(71) Applicant: ECO-BIOS., CO LTD., Jinju-si (KR)

(72) Inventor: Hang Hee CHO, Jinju-si (KR)

(73) Assignee: ECO-BIOS., CO LTD., Jinju-si (KR)

(21) Appl. No.: 18/457,768

(22) Filed: Aug. 29, 2023

(30) Foreign Application Priority Data
Nov. 17, 2022 (KR) 10-2022-0154750

(51) Int. Cl. A23L 33/135; A23L 29/275; A23L 29/281; A23L 29/30; A23L 31/00

(52) U.S. Cl. CPC A23L 33/135; A23L 29/275; A23L 29/284; A23L 29/30; A23L 31/00

(57) ABSTRACT

A lactobacillus fermentation composition includes mushroom extract, skim milk powder, fructooligosaccharide, gelatin hydrolyzate, and mixed lactobacillus strain.

### CROSS-REFERENCE TO RELATED APPLICATION

This application claims the benefit of Korean Patent Application No. 10-2022-0154750, filed on Nov. 17, 2022, in the Korean Intellectual Property Office, the disclosure of which is incorporated herein in its entirety by reference.

### BACKGROUND

### 1. Field

The present invention relates to a lactobacillus fermentation composition and a preparation method thereof, and more specifically, to a lactobacillus fermentation composition containing mushroom extract and gelatin hydrolyzate in order to enhance anticholesterol effects and bone health improvement effects, and a preparation method thereof.

### 2. Description of Related Art

Among food ingredients, many natural ingredients with lipid-improving effects and many natural ingredients with antioxidant effects have been reported. In particular, mushrooms, which are widely used for food and medicinal purposes, are attracting great attention as antioxidants. Lactobacillus fermented milk is also in the limelight as functional food for preventing aging and adult diseases through antioxidant action.

In addition, since lactobacillus fermented milk basically contains raw milk or skim milk powder as an additive, it is helpful in promoting bone health and promoting the growth of children and adolescents in the growing period and is also helpful for osteoporosis in the elderly and women.

Korean Patent Registration No. 10-0445685 discloses a lactobacillus medium composition containing mushroom powder or mushroom extract. This patent discloses a lactobacillus fermented mushroom composition that maintains or enhances pharmacological effects such as antioxidant action of mushrooms while increasing the fermentation rate of lactobacillus or enhancing pharmacological effects of lactobacillus.

However, the conventional invention has difficulty in increasing the pharmacological effect of mushrooms due to the low content of mushroom powder and also has limitations in promoting bone health only with casein phosphate peptide (CCP) contained in raw milk or skim milk powder.

### SUMMARY

An object to be achieved by the present invention is to propose a lactobacillus fermentation composition containing mushroom extract and gelatin hydrolyzate in order to enhance the pharmacological effects of mushrooms, gelatin, and lactobacillus.

In addition, another object to be achieved by the present invention is to propose a method for preparing a lactobacillus fermentation composition containing mushroom extract and gelatin hydrolyzate in order to enhance the pharmacological effects of mushrooms, gelatin, and lactobacillus.

A lactobacillus fermentation composition according to an embodiment of the present invention includes mushroom extract, skim milk powder, fructooligosaccharide, gelatin hydrolyzate, and mixed lactobacillus strain.

In addition, the lactobacillus fermentation composition includes 60-75 wt % of the mushroom extract, 3-20 wt % of the skim milk powder, 2-15 wt % of the fructooligosaccharide, 0.01-5 wt % of the gelatin hydrolyzate, and 0.1-10 wt % of the mixed lactobacillus strain.

In addition, the mushroom extract is extracted from one or more selected from the group consisting of shiitake mushroom, king oyster mushroom, and reishi mushroom.

In addition, the gelatin hydrolyzate is obtained by adding a proteolytic enzyme to gelatin.

In addition, the gelatin hydrolyzate is obtained by adding the gelatin in a weight ratio of 50 to 500 when a weight ratio of the proteolytic enzyme is 1.

In addition, the proteolytic enzyme is one or more selected from the group consisting of collagenase, gelatinase, alcalase, protamex, and flavorzyme.

In addition, the mixed lactobacillus strain is one or more selected from the group consisting of Lactobacillus acidophilus, Bifidobacterium longum, and Streptococcus thermophilus.

In addition, the lactobacillus fermentation composition further includes one or more selected from the group consisting of mushroom powder, chicory extract, natural carbohydrate, and purified water.

A method for preparing a lactobacillus fermentation composition according to an embodiment of the present invention includes: a step of producing mushroom extract; a step of producing gelatin hydrolyzate; a step of producing a lactobacillus medium by adding skim milk powder, fructooligosaccharide, and the gelatin hydrolyzate to the mushroom extract; a step of adding a mixed lactobacillus strain to the lactobacillus medium; a step of culturing the added mixed lactobacillus strain; and a step of maturing the cultured mixed lactobacillus strain.

In addition, the step of producing the mushroom extract includes adding one or more selected from the group consisting of shiitake mushroom, king oyster mushroom, and reishi mushroom to a high-pressure sterilizer and performing heating and cooling thereon.

### BRIEF DESCRIPTION DRAWINGS

FIG. 1 is a diagram schematically showing a method for preparing a lactobacillus fermentation composition of the present invention.

FIG. 2 is a diagram showing the weight of femur according to the feeding of the lactobacillus fermentation composition of the present invention.

FIG. 3 is a diagram showing the increase in bone strength (bar graph) and the mineral content of femur (line graph) according to the feeding of the lactobacillus fermentation composition of the present invention.

### DETAILED DESCRIPTION

Hereinafter, specific embodiments of the present disclosure will be described in detail with reference to the drawings. However, the spirit of the present invention is not limited to the presented embodiments, and those of ordinary skill in the art who understand the spirit of the present invention can easily suggest other degenerative inventions or other embodiments falling within the scope of the present invention through addition, change, deletion, and the like of other elements within the scope of the same spirit. However, this will also be said to fall within the scope of the present invention.

In addition, the terms as used herein are only used to describe specific embodiments, and are not intended to limit the present invention. The singular forms as used herein are intended to include the plural forms as well unless the context clearly indicates otherwise. In the specification of the present invention, the phrase “including a certain element” means “further including other elements” rather than excluding other elements unless otherwise stated.

Hereinafter, a lactobacillus fermentation composition of the present invention will be described.

The lactobacillus fermentation composition according to an embodiment of the present invention includes mushroom extract, skim milk powder, fructooligosaccharide, gelatin hydrolyzate, and mixed lactobacillus strain.

In addition, the lactobacillus fermentation composition may include 60-75 wt % of the mushroom extract, 3-20 wt % of the skim milk powder, 2-15 wt % of the fructooligosaccharide, 0.01-5 wt % of the gelatin hydrolyzate, and 0.1-10 wt % of the mixed lactobacillus strain.

In addition, the lactobacillus fermentation composition may further include one or more selected from the group consisting of mushroom powder, chicory extract, natural carbohydrate, and purified water.

In this case, 0.1-5 wt % of the mushroom powder, 2-5 wt % of the chicory extract, and 0.5-15 wt % of the natural carbohydrate may be included based on 100 wt % of the lactobacillus fermentation composition, and purified water may be the remainder based on 100 wt % of the lactobacillus fermentation composition.

For example, the natural carbohydrate may be one or more selected from the group consisting of monosaccharides such as glucose and fructose, disaccharides such as maltose and sucrose, polysaccharides such as dextrins and cyclodextrins, and sugar alcohols such as xylitol, sorbitol, and erythritol.

The lactobacillus fermentation composition according to an embodiment of the present invention may include 60-75 wt % of the mushroom extract, and preferably 65-70 wt % of the mushroom extract.

For example, edible mushroom and/or medicinal mushroom may be used as the mushroom extract, and fruiting bodies and/or mycelium may be used for the mushrooms. Preferably, the mushroom extract may be extracted from one or more selected from the group consisting of shiitake mushroom, king oyster mushroom, and reishi mushroom.

Here, the edible mushroom includes 70-95% of water and 5-30% of organic and inorganic components. Dried mushroom includes 15-30% of protein, 2-10% of fat, about 50% of a soluble inorganic material, and 5-10% of crude fiber, gallium, phosphoric acid, and ash.

In addition, it is known that the edible mushroom that generally has a good taste includes a lot of amino acid, mannite, trehalose, and the like, and also includes various vitamins and enzymes such as ergosterine, which is a precursor of vitamin B2 and vitamin D.

In addition, recently, mushrooms are known to be effective in suppressing aging and preventing and treating adult diseases, and thus, have anticancer effects, antimutagenic effects, serum lipid lowering effects, and immune enhancing effects. Accordingly, mushrooms are increasingly being used not only for food purposes but also for medicinal purposes. Mushrooms such as reishi mushroom, shiitake mushroom, king oyster mushroom, Phellinus linteus, Agaricus mushroom, ink mushroom, wood ear mushroom, horseshoe mushroom, and stone ear mushroom are known as mushrooms that are expected to have such physiological activity.

In particular, polysaccharide protein complex, which is a polysaccharide and protein complex contained in hot water extract as a medicinal ingredient of reishi mushroom, has been reported, and effects such as inhibition of cancer cell growth, treatment of essential hypertension, and inhibition of lipid peroxide production have been reported. Shiitake mushrooms, which are widely used for food purposes, are also widely used for medicinal purposes because shiitake mushrooms are effective in treating anticancer, cholesterol-lowering, tonic, diuretic, high blood pressure, nephritis, asthma, gastric ulcer, and the like. It has been reported that shiitake mushroom hot-water extract lowers lipids in serum and liver and inhibits liver damage. In addition, it has been reported that the polysaccharide extract of king oyster mushroom has serum cholesterol lowering effects, carbon tetrachloride-induced liver damage inhibitory effects, and antioxidant effects of king oyster mushroom fruiting body and mycelium extract.

Accordingly, the mushroom extract used in the present invention may be extracted from one or more selected from the group consisting of shiitake mushrooms, king oyster mushrooms, and reishi mushrooms, but the present invention is not limited thereto, and various modifications can be made thereto by those of ordinary skill in the art.

Subsequently, when shiitake mushroom and reishi mushroom are used in the mushroom extract, the reishi mushroom may be mixed in a weight ratio of 1 to 10 when a weight ratio of the shiitake mushroom is 1.

In addition, when the shiitake mushroom, the king oyster mushroom, and the reishi mushroom are used, the shiitake mushroom, the king oyster mushroom, and the reishi mushroom may preferably be mixed in a weight ratio of 0.5-5:0.5-5:0.5-3, and more preferably 4:4:2.

In addition, the lactobacillus fermentation composition according to an embodiment of the present invention contains a gelatin hydrolyzate, which is effective as a substance for preventing and treating bone diseases (for example, osteoporosis, fractures, and bone density loss) as well as a growth promoter for growing children.

Here, the gelatin used in the gelatin hydrolyzate may be used in animal epidermis, bone, intestines, and the like, and in particular, may be obtained from various livestock skins such as cow skin, pork skin, chicken skin, chicken wings, and chicken feet. Here, the pork skin is a food that has been consumed since ancient times, and main components of the pork skin are 61.48% of moisture, 34.44% of crude protein, 2.71% of crude fat, 1.42% of crude ash, and more than 70% of crude protein is collagen (Type I).

In addition, the collagen accounts for 30% of the protein in the human body as the main protein of connective tissue. In particular, the collagen is widely distributed in skin and bones and accounts for 70% of skin protein and 50% of cartilage. The collagen is present in the bones and skin of the human body, the membrane surrounding the internal organs, articular cartilage, and the cornea of the eye and particularly functions as an adhesive for calcium constituting bones.

In addition, the bone is a complex tissue and the main function of the bone is to resist physical force and breakage. Bone strength depends on the quality as well as the amount of bone tissue, which is characterized by the geometry and shape of the bone, the microstructure of cancellous bone, minerals, and collagen. In addition, other determinants of bone quality are closely linked to each other. In particular, minerals and collagen play a major role. Therefore, bone strength in front of physical force varies depending on a relationship between Type I collagen and minerals.

In addition, the human body has osteoblasts that form bones and osteoclasts that destroy bones. Osteoporosis may be caused when the rate of destruction of old or unnecessary bone, which is the process of bone resorption (osteoclasts), is faster than the rate of formation of new bone (osteoblasts, which make up and connect the outer cellular matrix of bone and are responsible for continual mineralization). In addition, there is a growing cartilage layer, which is an aggregate of chondrocytes, at both ends of the bones of growing children and adolescents. When this becomes the epiphyseal end, osteoblasts are formed and transformed into bones, thereby growing in the vertical direction and increasing height.

In addition, the gelatin hydrolyzate may be obtained by adding a proteolytic enzyme to gelatin.

For example, the proteolytic enzyme may be one or more selected from the group consisting of collagenase, gelatinase, alcalase, protamex, and flavorzyme. Preferably, protamex and flavozyme may be selected.

In addition, the gelatin hydrolyzate may be added in a weight ratio of 50 to 500 when a weight ratio of the proteolytic enzyme is 1. Preferably, the gelatin may be added in a weight ratio of 100 when a weight ratio of the proteolytic enzyme is 1.

In addition, although the appropriate reaction time, appropriate temperature, and appropriate pH of the proteolytic enzyme may vary depending on characteristics of each enzyme. Preferably, it may be carried out under conditions of a reaction time of 2-24 hours, a temperature of 30-60° C., and a pH of 6.0-9.0.

In addition, after the gelatin hydrolyzate is desalted, the gelatin hydrolyzate may be separated and purified by a method including ultrafiltration, gel filtration, various types of chromatography, membrane filter, and isoelectric point before use.

For example, the gelatin hydrolyzate may be separated into a size of 50 kDa or less, 50 kDa to 3 kDa, or 3 kDa or less by using a membrane filter. At this time, when the gelatin hydrolyzate is divided into decomposition time, type of processing enzyme, and molecular weight, the activity according to each fraction may show various aspects depending on the type and state of the protein contained in the fraction. A composition containing some fraction of the gelatin hydrolyzate obtained in this manner as an active ingredient may be effective as a substance for preventing and treating bone diseases as well as a growth promoter for growing children.

In addition, the lactobacillus fermentation composition according to an embodiment of the present invention is characterized by including a mixed lactobacillus strain. The mixed lactobacillus strain may be one or more selected from the group consisting of Lactobacillus acidophilus, Bifidobacterium longum, and Streptococcus thermophilus, but the present invention is not limited thereto, various modifications can be made thereto by those of ordinary skill in the art.

For example, as bacteria belonging to lactobacillus, there are dozens of species such as streptococcus, pediococcus, leuconostoc, lactobacillus, and bifidobacterium. The lactobacillus is widely distributed in the natural world, including human and animal digestive tracts or dozens of agricultural products. Bulgarian bacteria, yogurt bacteria, and thermophilus bacteria are used to prepare yogurt. Yogurt bacteria, casei bacteria, and acidophilus bacteria are used to prepare lactobacillus beverages. Casei bacteria, cremoris bacteria, Heberikis bacteria, and milk streptococci are used to prepare cheese. Milk streptococci is also used in fermented butter. As described above, certain types of lactobacillus are involved in each preparation in the food processing process.

Here, lactobacillus is attached to intestinal epithelial cells and performs metabolic activities to suppress harmful bacteria by secreting lactic acid, (low-grade) fatty acid, antibiotics (bacteriocin), H₂O₂, and the like. Cholesterol production by HMG (hydroxy methyl glutaric), orotic acid, uric acid, and the like prepared by lactobacillus fermentation is inhibited. In particular, Lactobacillus acidophilus, which is a type of lactobacillus, directly decomposes cholesterol. In addition, lactobacillus rapidly detects bacteria and viruses through the activation of microphages that detect pathogens in an immune system, prevents cancer cell proliferation by promoting lymphocyte division, increases the production of IgA, which is an antibody in blood, improves immunity by enhancing the production of gamma-interferon, increases the nutritional value of food, acts to inhibit endogenous infections, and induces the growth inhibition and death of harmful bacteria that produce carcinogens in the intestines to thereby have anticancer effects.

Hereinafter, a detailed description of the method for preparing a lactobacillus fermentation composition may be omitted to the extent that it overlaps the above description.

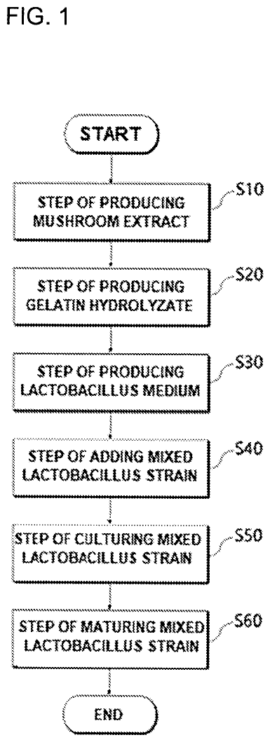

Referring to FIG. 1, the method for preparing a lactobacillus fermentation composition according to an embodiment of the present invention may include: a step S10 of producing a mushroom extract; a step S20 of producing a gelatin hydrolyzate; a step S30 of producing a lactobacillus medium by adding skim milk powder, fructooligosaccharide, and gelatin hydrolyzate to the mushroom extract; a step S40 of adding a mixed lactobacillus strain to the lactobacillus medium; a step S50 of culturing the added mixed lactobacillus; and a step S60 of maturing the cultured mixed lactobacillus strain.

1) The step S10 of producing the mushroom extract includes putting one or more selected from the group consisting of shiitake mushroom, king oyster mushroom, and reishi mushroom into a high-pressure sterilizer, adding purified water in a weight ratio of 8 to 10 when a weight ratio of the mushroom is 1, performing heating for 1-3 hours at a temperature of 100-130° C., and performing primary cooling.

2) Next, the primarily cooled mushroom is heated again at a temperature of 70-100° C. for 1-3 hours and is secondarily cooled.

3) Finally, the secondarily cooled mushroom is heated again at a temperature of 50-70° C. for 1-3 hours and is then left at room temperature for 4-8 hours and performs tertiary cooling until a final temperature drops below 30° C.

As described above, the fruiting body content of the mushroom extract may be improved through not the primary cooling but the primary, secondary, and tertiary cooling, thereby greatly improving the pharmacological effect of the mushroom.

Next, 1) the step S20 of producing the gelatin hydrolyzate adjusting pH of a gelatin solution to 6.0 to 9.0 and adding a proteolytic enzyme in a weight ratio of 0.002 to 0.02 when a weight ratio of the gelatin solution is 1.

2) Next, desalting is performed by hydrolyzing the gelatin solution, to which the proteolytic enzyme is added, at a temperature of 30-60° C. for a reaction time of 2-24 hours.

3) Next, the desalted gelatin hydrolyzate is separated into a predetermined size (e.g., 50 kDa or less) through at least one of the methods including ultrafiltration, gel filtration, various types of chromatography, membrane filter, and isoelectric point, thereby obtaining a gelatin hydrolyzate having a size equal to or less than the predetermined size.

4) The obtained gelatin hydrolyzate is lyophilized.

Next, 1) the step S30 of producing the lactobacillus medium includes mixing skim milk powder, fructooligosaccharide, and the gelatin hydrolyzate with the mushroom extract and homogenizing the mixture.

2) Next, the homogenized mixture is heated at a temperature of 80-120° C. for 0.1-2 hours and then cooled to 40° C. or less.

Subsequently, the step S40 of adding the mixed lactobacillus strain includes adding the mixed lactobacillus strain to the lactobacillus medium cooled to 40° C. or less. Preferably, when the temperature of the lactobacillus medium is 37-39° C., the mixed lactobacillus strain is added.

Next, the step S50 of culturing the mixed lactobacillus strain includes culturing the added mixed lactobacillus strain in an incubator at 34-40° C. for 5-18 hours.

Finally, the step S60 of maturing the mixed lactobacillus strain includes maturing the cultured mixed lactobacillus strain for 10-24 hours at a temperature of 3-5° C.

Hereinafter, experimental examples and/or examples of the present invention will be described so that those of ordinary skill in the art can easily carry out the present invention. However, the present invention may be implemented in various different forms and is not limited to the experimental examples and/or the examples described herein.

TABLE 1

Content (unit: wt %)

| | | | Comparative |
|---|---|---|---|
| No. | Name of raw material | Example 1 | Example 1 |
| 1 | Skim milk powder | 20.0 | 10.0 |
| 2 | Raw milk | — | 80.7 |
| 3 | Fructooligosaccharide | 9.0 | — |
| 4 | Sugar | — | 9.0 |
| 5 | Mushroom extract | 70.0 | — |
| 6 | Gelatin hydrolyzate | 0.7 | — |
| 7 | Lactobacillus acidophilus | 0.3 | 0.3 |

| | | |
|---|---|---|
| Total | 100.00 | 100.00 |

### Example

A lactobacillus fermentation composition of Example 1 was prepared by mixing the raw materials listed in Table 1 above.

Preparation Method

1) Shiitake mushroom, king oyster mushroom, and reishi mushroom were added to a high-pressure sterilizer at a ratio of 4:4:2, and purified water was added in a weight ratio of 10 when a total weight ratio of the mushroom was 1, was heated at 120° C. for 2 hours, and was then primarily cooled.

2) The primarily cooled mushroom was heated again at a temperature 85° C. for 2 hours and was secondarily cooled.

3) The secondarily cooled mushroom was heated again at a temperature 60° C. for 2 hours and was then left at room temperature for 6 hours and performed tertiary cooling until a final temperature dropped below 30° C. In this manner, a mushroom extract was produced.

4) pH of the gelatin solution was adjusted to 7.0, and a proteolytic enzyme including protamex and flavozyme was added in a weight ratio of 0.01 when a weight ratio of the gelatin solution was 1. Desalting was performed by hydrolysis at 50° C. for 12 hours. A gelatin hydrolyzate of 3 kDa or less was obtained by using a membrane filter and was then lyophilized.

5) Skim milk powder, fructooligosaccharide, and the gelatin hydrolyzate were mixed with the produced mushroom extract, and the mixture was homogenized.

6) The homogenized mixture (lactobacillus medium) was heated at 100° C. for 1 hour and then cooled to 37° C.

7) Lactobacillus acidophilus was added to the mixture (lactobacillus medium) cooled to 37° C., and the lactobacillus was cultured in an incubator at 37° C. for 8 hours.

8) Finally, the mixture (lactobacillus medium) in which the lactobacillus was cultured was matured at 4° C. for 15 hours.

### Comparative Example

Common plain yogurt of Comparative Example 1 was prepared by mixing the raw materials listed in Table 1 above.

Preparation Method

1) Skim milk powder, sugar, and milk were mixed and homogenized.

2) The homogenized mixture (lactobacillus medium) was heated at 100° C. for 1 hour and then cooled to 37° C.

3) Lactobacillus acidophilus was added to the mixture (lactobacillus medium) cooled to 37° C., and the lactobacillus was cultured in an incubator at 37° C. for 8 hours.

4) Finally, the mixture (lactobacillus medium) in which the lactobacillus was cultured was matured at 4° C. for 15 hours.

### EXPERIMENTAL EXAMPLES

### Experimental Example 1: Comparison of the Number of Lactobacilli

In order to examine the degree of lactobacillus growth in the lactobacillus medium compositions of Example and Comparative Example, the lactobacillus was cultured anaerobically at 37° C. for 16 hours in the two mediums. After 3% of Bifidobacterium longum KCTC8649P spawn was inoculated, the Erlenmeyer flask space was replaced with CO₂ from which O₂ was removed, and the spawn was cultured anaerobically at 37° C. for 16 hours.

100 μl of each culture solution was diluted with a diluent and plated on the medium to form 30-300 colonies per plate. Then, the number of colonies obtained by culturing in an anaerobic incubator at 37° C. for 48 hours was counted and the number of viable cells was calculated by multiplying a dilution factor, and the results thereof are shown in Table 2 below.

TABLE 2

| Classification | Lactobacillus count (CFU/g) |
|---|---|
| Example 1 | 5,750,000,000,000 |
| Comparative Example 1 | 7,900,000,000 |

It can be seen from Table 2 that the lactobacillus culture composition of Example according to the present invention proliferates lactobacillus at a high concentration much more excellent than the conventional comparative example.

### Experimental Example 2: Anticholesterol Effect

Nine 6-week-old male ddY rats were used as experimental animals. The experimental rats were classified into three groups: a normal diet group, a lactobacillus fermentation composition diet group according to Example 1, and a lactobacillus fermentation composition diet group according to Comparative Example 1, and the lactobacillus fermentation composition was added at a level of 15% in the diet and the experimental rats were bred for 3 weeks.

After 3 weeks, blood was collected from the aorta of the rats in the three groups, and the cholesterol concentration in the blood was quantified by using a cholesterol Wako Kit (Wako Junyaku, Osaka, Japan), and the results thereof are shown in Table 3 below.

TABLE 3

| | | | | | | | Diet group of | | |
|---|---|---|---|---|---|---|---|---|---|
| | Normal diet group | | | Diet group of Example 1 | | | Comparative Example 1 | | |
| | First | Second | Third | Fourth | Fifth | Sixth | Seventh | Eighth | Ninth |
| Classification | mouse | mouse | mouse | mouse | mouse | mouse | mouse | mouse | mouse |
| Total cholesterol (ug/ml) | 620.2 | 680.5 | 630.8 | 655.5 | 653.2 | 670.4 | 736.4 | 733.2 | 750.2 |
| HDL-cholesterol (ug/ml) | 441.5 | 451.2 | 445.4 | 477.5 | 473.5 | 480.2 | 438.9 | 436.5 | 445.6 |
| Triglyceride (ug/ml) | 1330 | 1358 | 1332 | 1106 | 1005 | 1112 | 1268 | 1255 | 1290 |
| Phospholipid (ug/ml) | 950 | 1005 | 998 | 966 | 952 | 968 | 963 | 943 | 980 |

Table 3 shows cholesterol concentrations in the blood. In the experimental rat group bred with the lactobacillus fermentation composition according to Example 1, the HDL-cholesterol concentration in the blood increased, whereas the concentrations of triglyceride and phospholipid decreased, compared to the normal diet group and the experimental rat group bred by the lactobacillus fermentation composition according to Comparative Example 1 This indicates that the mushroom lactobacillus fermentation composition according to the present invention increases the HDL-cholesterol concentration, compared to general lactobacillus fermentation compositions, thereby enhancing physiological activity, and it can be seen that the lactobacillus cultured in the lactobacillus fermentation composition of the present invention has an excellent anticholesterol effect.

### Experimental Example 3: Bone Health Promotion Effect

As experimental animals, 6-week-old Sprague-Dawley rats were used for evaluation. The experimental rats were classified into three groups: a normal diet group, a lactobacillus fermentation composition diet group according to Example 1, and a lactobacillus fermentation composition diet group according to Comparative Example 1, and the lactobacillus fermentation composition was fed for 7 weeks at 0.5 mL each.

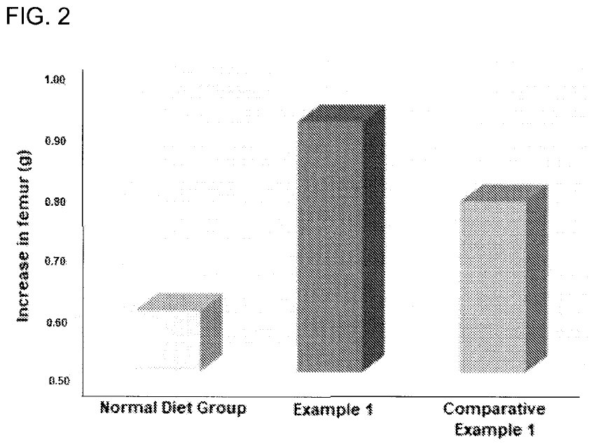

Referring to FIG. 2, the amount of increase in the femur of the normal diet group was 0.59 g, the amount of increase in the femur of Example 1 was 0.91 g, and the amount of increase in the femur of Comparative Example 1 was 0.78 g. It was confirmed that the amount of increase in the femur of Example 1 was increased by about 35% compared to the normal diet group and by about 14% compared to Comparative Example 1.

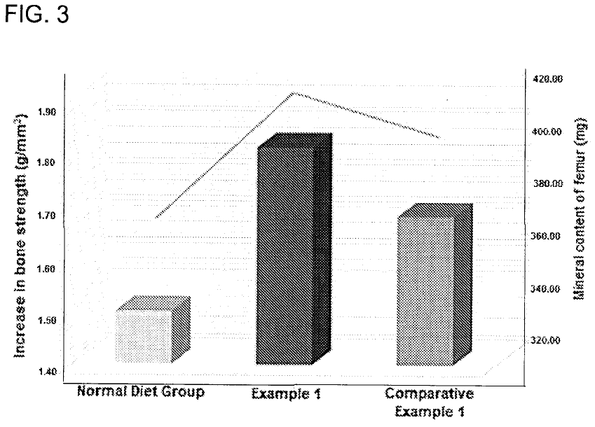

Referring to FIG. 3, the mineral content of the femur in the normal diet group was 362.83 mg, the mineral content of the femur in Example 1 was 415.78 mg, and the mineral content of the femur in Comparative Example 1 was 394.52 mg. It was confirmed that the mineral content of the femur in Example 1 was increased by about 13% compared to the normal diet group and by about 5% compared to Comparative Example 1. In addition, the increase in the mineral content of the femur reflected the increase in bone strength, and it was confirmed that the increase in bone strength of Example 1, which has a high mineral content, was great compared to the normal diet group and Comparative Example 1.

### Experimental Example 4: Sensory Evaluation

Sensory evaluation was performed on the pine mushroom yogurt prepared according to the Example and the commercially available yogurt according to the Comparative Example.

For the sensory evaluation method, each yogurt was given to 50 men and women in their teens to 40s, and the color, richness, flavor, sweetness, and overall preference were measured using a 5-point scoring method (5 points; very good 4 points: good 3 points; average 2 points; bad 1 point; very bad), and the average value was obtained. The results thereof are shown in Table 4 below.

TABLE 4

| | | | | | Overall |
|---|---|---|---|---|---|
| | Color | Richness | Flavor | Sweetness | preference |
| Example 1 | 4.3 | 4.2 | 4.0 | 4.4 | 4.2 |
| Comparative | 4.2 | 4.1 | 4.1 | 4.2 | 4.1 |
| Example 1 | | | | | |

Referring to Table 4, as a result of comparing the overall preference of the lactobacillus fermentation composition according to Example 1 with the general lactobacillus fermentation composition according to Comparative Example 1, it was confirmed that there was no significant difference therebetween. From this, it was confirmed that the problem of preference did not occur when recommending intake to growing adolescents.

In order to more clearly express the technical spirit of the present invention, components that have no relevance to the technical spirit of the present invention are briefly expressed or omitted in the accompanying drawings.

In a lactobacillus fermentation composition and a preparation method thereof, a lactobacillus medium containing mushroom extract and gelatin hydrolyzate is produced, and a mixed lactobacillus strain is added to the lactobacillus medium and then cultured and matured. Accordingly, the lactobacillus fermentation composition has pharmacological effects, such as antithrombotic and antilipidic action, antibacterial action, anticancer action, antidiabetic action, or antioxidant action of mushrooms and lactobacillus, and bone health improvement effects of gelatin hydrolyzate.

However, the effects of the present invention are not limited to those described above, and the effects not mentioned herein will be clearly understood from the present specification and the accompanying drawings by those of ordinary skill in the art.

Although the configurations and features of the present invention have been described with reference to the embodiments of the present invention, the present invention is not limited thereto, and it will be apparent to those of ordinary skill in the art that various changes or modifications can be made thereto without departing from the spirit and scope of the present invention. Therefore, it is noted that these changes or modifications will fall within the scope of the appended claims.

### DESCRIPTION OF SYMBOLS

S10: Step of producing mushroom extract
S20: Step of producing gelatin hydrolyzate
S30: Step of producing lactobacillus medium
S40: Step of adding mixed lactobacillus strain
S50: Step of culturing mixed lactobacillus strain
S60: Step of maturing mixed lactobacillus strain

What is claimed is:

1. A lactobacillus fermentation composition comprising mushroom extract, skim milk powder, fructooligosaccharide, gelatin hydrolyzate, and mixed lactobacillus strain.

2. The lactobacillus fermentation composition of claim 1, wherein the lactobacillus fermentation composition includes 60-75 wt % of the mushroom extract, 3-20 wt % of the skim milk powder, 2-15 wt % of the fructooligosaccharide, 0.01-5 wt % of the gelatin hydrolyzate, and 0.1-10 wt % of the mixed lactobacillus strain.

3. The lactobacillus fermentation composition of claim 1, wherein the mushroom extract is extracted from one or more selected from the group consisting of shiitake mushroom, king oyster mushroom, and reishi mushroom.

4. The lactobacillus fermentation composition of claim 1, wherein the gelatin hydrolyzate is obtained by adding a proteolytic enzyme to gelatin.

5. The lactobacillus fermentation composition of claim 4, wherein the gelatin hydrolyzate is obtained by adding the gelatin in a weight ratio of 50 to 500 when a weight ratio of the proteolytic enzyme is 1.

6. The lactobacillus fermentation composition of claim 4, wherein the proteolytic enzyme is one or more selected from the group consisting of collagenase, gelatinase, alcalase, protamex, and flavorzyme.

7. The lactobacillus fermentation composition of claim 1, wherein the mixed lactobacillus strain is one or more selected from the group consisting of Lactobacillus acidophilus, Bifidobacterium longum, and Streptococcus thermophilus.

8. The lactobacillus fermentation composition of claim 1, wherein the lactobacillus fermentation composition further comprises one or more selected from the group consisting of mushroom powder, chicory extract, natural carbohydrate, and purified water.

9. A method for preparing the lactobacillus fermentation composition of claim 1, the method comprising:
a step of providing a lactobacillus medium comprising skim milk powder, a fructooligosaccharide, and a gelatin hydrolysate, and a mushroom extract;
a step of adding a mixed lactobacillus strain to the lactobacillus medium;
a step of culturing the added mixed lactobacillus strain; and
a step of maturing the cultured mixed lactobacillus strain.

10. The method of claim 9, wherein the step of producing the mushroom extract comprises adding one or more selected from the group consisting of shiitake mushroom, king oyster mushroom, and reishi mushroom to a high-pressure sterilizer and performing heating and cooling thereon.
